(19) United States
(12) Patent Application Publication
(10) Pub. No.: US 2006/0219242 A1
(43) Pub. Date: Oct. 5, 2006

(54) Method for the Administration of an Anticholinergic by Inhalation

(75) Inventor: Bernd Zierenberg, Bingen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International, Ingelheim (DE)

(21) Appl. No.: 11/277,886

(22) Filed: Mar. 29, 2006

Related U.S. Application Data
(60) Provisional application No. 60/666,428, filed on Mar. 30, 2005.

(51) Int. Cl. A61M 11/00; A61K 9/14; A61K 31/4745

(52) U.S. Cl. 128/200.23; 424/46; 514/291

(57) ABSTRACT

The invention relates to a receptacle comprising tiotropium or a physiologically acceptable salt thereof and a physiologically acceptable excipient, wherein the receptacle is able to store the tiotropium under conditions that prevent moisture uptake.

### RELATED APPLICATIONS

This application claims priority to U.S. provisional application 60/666,428, filed Mar. 30, 2005, the content of which is incorporated herein by reference in its entirety.

### FIELD OF THE INVENTION

The invention relates to a method for the administration of powdered preparations containing tiotropium by inhalation.

### BACKGROUND OF THE INVENTION

Tiotropium bromide is known from European Patent Application EP 418 716 A1 and has the following chemical structure:

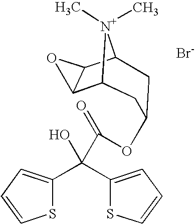

Tiotropium bromide is a highly effective anticholinergic with a long-lasting activity which can be used to treat respiratory complaints, particularly COPD (chronic obstructive pulmonary disease) and asthma. The term tiotropium refers to the free ammonium cation.

For treating the abovementioned complaints, it is useful to administer the active substance by inhalation. In addition to the administration of broncholytically active compounds in the form of metered aerosols and inhalable solutions, the use of inhalable powders containing active substance is of particular importance.

With active substances which have a particularly high efficacy, only small amounts of the active substance are needed per single dose to achieve the desired therapeutic effect. In such cases, the active substance has to be diluted with suitable excipients in order to prepare the inhalable powder. Because of the large amount of excipient, the properties of the inhalable powder are critically influenced by the choice of excipient. When choosing the excipient its particle size is particularly important. As a rule, the finer the excipient, the poorer its flow properties. However, good flow properties are a prerequisite for highly accurate metering when packing and dividing up the individual doses of preparation, e.g. when producing capsules for powder inhalation or when the patient is metering the individual dose before using a multi-dose inhaler. It has also been found that the particle size of the excipient has a considerable influence on the proportion of active substance in the inhalable powder which is delivered for inhalation. The term inhalable proportion of active substance refers to the particles of the inhalable powder which are conveyed deep into the branches of the lungs when inhaled with a breath. The particle size required for this is between 1 and 10 μm, preferably less than 5 μm.

Finally, it has been found that the intended therapeutic effect upon the administration of a pharmaceutical composition via inhaltion can be decisively influenced by the inhalation device.

Accordingly, the aim of the invention is to provide for a therapeutically efficient method for the administration of inhalable powders containing tiotropium. Another object of the invention is to provide for an inhalation kit comprising a tiotropium containing powder and an inhalation device, said kit being applicable in the method for administration mentioned before.

### DETAILED DESCRIPTION OF THE INVENTION

In the method according to the invention an inhalable powder containing tiotropium, preferably in an amount of 0.001 to 5%, in admixture with a physiologically acceptable excipient is administered.

Of particular interest for the method according to the invention is an inhalable powder containing 0.01 to 2%, preferably 0.04 to 0.8%, more preferably 0.08 to 0.64% tiotropium in admixture with a physiologically acceptable excipient is administered. More preferably in the method according to the invention an inhalable powder containing 0.1 to 0.4% tiotropium in admixture with a physiologically acceptable excipient is administered.

By tiotropium is meant the free ammonium cation. The counter-ion (anion) may be chloride, bromide, iodide, methanesulphonate, para-toluenesulphonate or methyl sulphate. Of these anions, the bromide is preferred.

Accordingly, the method according to the present invention preferably relates to inhalable powders which contain tiotropium in form of tiotropium bromide in an amount of 0.0012 to 6.02%, in admixture with a physiologically acceptable excipient. Of particular interest for the method according to the invention is an inhalable powder containing 0.012 to 2.41%, preferably 0.048 to 0.96%, more preferably 0.096 to 0.77% tiotropium bromide in admixture with a physiologically acceptable excipient is administered.

More preferably in the method according to the invention an inhalable powder containing 0.12 to 0.48% tiotropium bromide in admixture with a physiologically acceptable excipient is administered.

Tiotropium bromide is, depending on the choice of reaction conditions and solvents, obtainable in different crystalline modifications. Most preferred according to the invention are those powder preparations, that contain tiotropium in form of the crystalline tiotropium bromide monohydrate. Accordingly, the powdered preparations obtainable according to the invention preferably contain 0.0012 to 6.25% crystalline tiotropium bromide monohydrate in admixture with a physiologically acceptable excipient is administered. Of particular interest for the method according to the invention is an inhalable powder containing 0.0125 to 2.5%, preferably 0.05 to 1%, more preferably 0.1 to 0.8% crystalline tiotropium bromide monohydrate in admixture with a physiologically acceptable excipient is administered.

More preferably in the method according to the invention an inhalable powder containing 0.12 to 0.5% crystalline tiotropium bromide monohydrate in admixture with a physiologically acceptable excipient is administered.

Examples of physiologically acceptable excipients which may be used to prepare the inhalable powders applicable according to the invention include, for example, monosaccharides (e.g. glucose or arabinose), disaccharides (e.g. lactose, saccharose, maltose), oligo- and polysaccharides (e.g. dextrane), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates, preferably in the form of their monohydrates.

In the method according to the invention the average particle size of the physiologically acceptable excipient is preferably between 10 to 500 μm, more preferably between 15 to 200 μm, most preferably between 20 to 100 μm. If not otherwise emphazised the term average particle size according to the invention is to be understood as the Mass Median Aerodynamic Diameter (MMAD). Methods for the determination thereof are known in the art.

Besides the coarser particle fraction of the excipient mentioned hereinbefore, the excipient can optionally additionally contain a specifically added fraction of excipient of finer particle size. This finer particle size fraction is characterized by an average particle size of 1 to 9 μm, preferably 2 to 8 μm, more preferably 3 to 7 μm. If a finer particle fraction is present the proportion of finer excipient in the total amount of excipient is 1 to 20%, preferably 3 to 15%, more preferably 5 to 10%. When reference is made to a mixture within the scope of the present invention, this always means a mixture obtained by mixing together clearly defined components. Accordingly, when an excipient mixture of coarser and finer excipients is mentioned, this can only denote mixtures obtained by mixing a coarser excipient component with a finer excipient component.

The percentages given within the scope of the present invention are always percent by weight.

In the method according to the invention the inhalable powders mentioned hereinbefore may effeciently be adminstered using inhalers that are characterized by a specific flow resistance (R).

The flow resistance of inhalers can be calculated via the following formula:

$$V = \frac{1}{R} \cdot \sqrt{p}$$

wherein v is the volumetric flow rate (l/min),

p is the pressure drop (kPa), and

R is the flow resistance.

In the method according to the invention the flow resistance R characterising the inhaler is in a range of about 0.01-0.1 \sqrt{kPa} min/l preferably in the range of about 0.02-0.06 \sqrt{kPa} min/l.

Accordingly, the invention relates to a method for the administration of an inhalable powder containing tiotropium, preferably in an amount of 0.001 to 5%, in admixture with a physiologically acceptable excipient with an average particle size of between 10 to 500 μm, and further characterized in that the said tiotropium containing powder is administered by an inhaler displaying a flow resistance of about 0.01-0.1 \sqrt{kPa} min/l.

In another embodiment, the invention relates to a method for the treatment of airway diseases, particularly COPD (chronic obstructive pulmonary disease) and asthma, characterized in that an inhalable powder containing tiotropium, preferably in an amount of 0.001 to 5%, in admixture with a physiologically acceptable excipient with an average particle size of between 10 to 500 μm, is administered via inhalation by an inhaler displaying a flow resistance of about 0.01-0.1 \sqrt{kPa}·min/l.

In another embodiment the invention relates to the use of an inhaler for the administration of a tiotropium containing inhalable powder via inhalation, characterised in that the inhalable powder contains tiotropium, preferably in an amount of 0.001 to 5%, in admixture with a physiologically acceptable excipient with an average particle size of between 10 to 500 μm, and further characterized in that the said inhaler displays a flow resistance of about 0.01-0.1 \sqrt{kPa} min/l.

In yet another embodiment the invention relates to an inhalation kit consisting of an inhaler displaying a flow resistance of about 0.01-0.1 \sqrt{kPa} min/l and an inhalable powder containing tiotropium, preferably in an amount of 0.001 to 5%, in admixture with a physiologically acceptable excipient with an average particle size of between 10 to 500 μm.

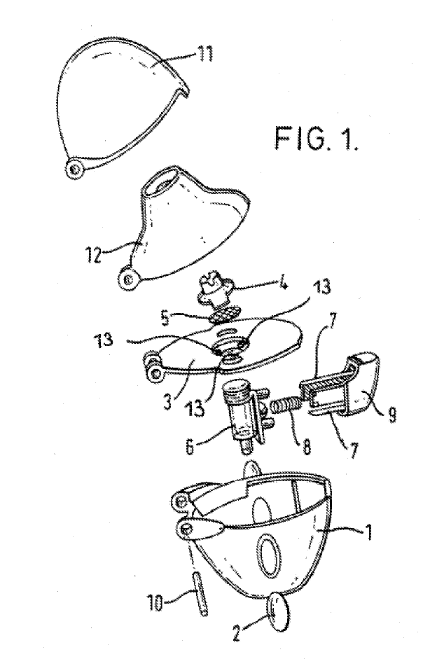

In another preferred embodiment according to the invention the inhaler described in FIG. 1 is applied. For the administration of tiotropium containing powders by inhalation by means of the inhaler according to FIG. 1, it is required to fill appropriate amounts of the powder into capsules. Methods for filling powders into capsules are known in the art.

The inhaler according to FIG. 1 is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, a mouthpiece 12 which is connected to the housing 1, the deck 3 and a cover 11 via a spindle 10 to enable it to be flipped open or shut and three holes 13 with diameters below 1 mm in the central region around the capsule chamber 6 and underneath the screen housing 4 and screen 5.

The main air flow enters the inhaler between deck 3 and base 1 near to the hinge. The deck has in this range a reduced width, which forms the entrance slit for the air. Then the flow reverses and enters the capsule chamber 6 through the inlet tube. The flow is then further conducted through the filter and filter holder to the mouthpiece. A small portion of the flow enters the device between mouthpiece and deck and flows then between filterholder and deck into the main stream. Due to production tolerances there is some uncertainty in this flow because of the actual width of the slit between filterholder and deck. In case of new or reworked tools the flow resistance of the inhaler may therefore be a little off the target value. To correct this deviation the deck has in the central region around the capsule chamber 6 and underneath the screen housing 4 and screen 5 three holes 13 with diameters below 1 mm. Through these holes 13 flows air from the base into the main air stream and reduces such slightly the flow resistance of the inhaler. The actual diameter of these holes 13 can be chosen by proper inserts in the tools so that the mean flow resistance can be made equal to the target value.

Accordingly, in a preferred embodiment the invention relates to a method for the administration of an inhalable powder containing tiotropium, preferably in an amount of 0.001 to 5%, in admixture with a physiologically acceptable excipient with an average particle size of between 10 to 500 μm, by means of the inhaler according to FIG. 1, comprising

a housing, containing two windows, a deck in which there are air inlet ports and which is provided with a screen secured via a screen housing, an inhalation chamber connected to the deck on which there is a push button provided with two sharpened pins and movable counter to a spring, a mouthpiece which is connected to the housing, the deck and a cover via a spindle to enable it to be flipped open or shut, and three holes with diameters below 1 mm in the central region around the capsule chamber and underneath the screen housing and screen.

In another embodiment, the invention relates to a method for treatment of airway diseases, particularly COPD (chronic obstructive pulmonary disease) and asthma, charcterized in that an inhalable powder containing tiotropium, preferably in an amount of 0.001 to 5%, in admixture with a physiologically acceptable excipient with an average particle size of between 10 to 500 μm, is administered via inhalation by the inhaler according to FIG. 1, comprising

a housing, containing two windows, a deck in which there are air inlet ports and which is provided with a screen secured via a screen housing, an inhalation chamber connected to the deck on which there is a push button provided with two sharpened pins and movable counter to a spring, a mouthpiece which is connected to the housing, the deck and a cover via a spindle to enable it to be flipped open or shut, and three holes with diameters below 1 mm in the central region around the capsule chamber and underneath the screen housing and screen.

In another preferred embodiment the invention relates to the use of the inhaler according to FIG. 1, comprising a housing, containing two windows,

a deck in which there are air inlet ports and which is provided with a screen secured via a screen housing, an inhalation chamber connected to the deck on which there is a push button provided with two sharpened pins and movable counter to a spring;

a mouthpiece which is connected to the housing, the deck and a cover via a spindle to enable it to be flipped open or shut, and three holes with diameters below 1 mm in the central region around the capsule chamber and underneath the screen housing and screen,

for the administration of an inhalable powdered containing tiotropium, preferably in an amount of 0.001 to 5%, in admixture with a physiologically acceptable excipient with an average particle size of between 10 to 500 μm.

In yet another preferred embodiment the invention relates to an inhalation kit consisting of an inhalable powdered containing tiotropium, preferably in an amount of 0.001 to 5%, in admixture with a physiologically acceptable excipient with an average particle size of between 10 to 500 μm, and the inhaler according to FIG. 1, comprising

a housing, containing two windows, a deck in which there are air inlet ports and which is provided with a screen secured via a screen housing, an inhalation chamber connected to the deck on which there is a push button provided with two sharpened pins and movable counter to a spring, a mouthpiece which is connected to the housing, the deck and a cover via a spindle to enable it to be flipped open or shut, and three holes with diameters below 1 mm in the central region around the capsule chamber and underneath the screen housing and screen.

In another preferred embodiment according to the invention the inhaler according to U.S. Pat. No. 4,524,769 is applied. This inhaler (or inhalator) is activated by the air flow generated at inhalation. The disclosure of U.S. Pat. No. 4,524,769 is incorporated herein by reference in its entirety.

Accordingly, in a preferred embodiment the invention relates to a method for the administration of an inhalable powder containing tiotropium, preferably in an amount of 0.001 to 5%, in admixture with a physiologically acceptable excipient with an average particle size of between 10 to 500 μm, by means of the inhaler according to U.S. Pat. No. 4,524,769, comprising a nozzle, a conduit connected to the nozzle, a storage chamber adjacent said conduit for storing said inhalable powder to be dispensed by said inhalator, a perforated membrane having a plurality of preselected perforated portions each holding and dispensing a reproducible unit dose of less than 50 mg of the said inhalable pwder, said membrane being mounted for movement between said conduit and said storage chamber so that one of said preselected portions is positioned across said conduit whereby the active compound held in the perforation thereof can be dispensed into the conduit and another of said preselected portions thereof is disposed within said storage chamber,

dose loading means for introducing said inhalable powder in the storage chamber into the perforation of the preselected portion of said membrane disposed within the storage chamber, and maneuvering means for displacing the perforated membrane through a plurality of positions whereby successive preselected portions of the perforated membrane holding the inhalable powder are positioned across said conduit for dispensing the inhalable powder.

In another embodiment, the invention relates to a method for treatment of airway diseases, particularly COPD (chronic obstructive pulmonary disease) and asthma, charcterized in that an inhalable powder containing tiotropium, preferably in an amount of 0.001 to 5%, in admixture with a physiologically acceptable excipient with an average particle size of between 10 to 500 μm, is administered via inhalation by the inhaler according to U.S. Pat. No. 4,524,769, comprising

a nozzle, a conduit connected to the nozzle, a storage chamber adjacent said conduit for storing said inhalable powder to be dispensed by said inhalator,

a perforated membrane having a plurality of preselected perforated portions each holding and dispensing a reproducible unit dose of less than 50 mg of the said inhalable powder, said membrane being mounted for movement between said conduit and said storage chamber so that one of said preselected portions is positioned across said conduit whereby the active compound held in the perforation thereof can be dispensed into the conduit and another of said preselected portions thereof is disposed within said storage chamber, dose loading means for introducing said inhalable powder in the storage chamber into the perforation of the preselected portion of said membrane disposed within the storage chamber, and

maneuvering means for displacing the perforated membrane through a plurality of positions whereby successive preselected portions of the perforated membrane holding the inhalable powder are positioned across said conduit for dispensing the inhalable powder.

In another preferred embodiment the invention relates to the use of the inhaler according to U.S. Pat. No. 4,524,769 comprising

a nozzle, a conduit connected to the nozzle, a storage chamber adjacent said conduit for storing said inhalable powder to be dispensed by said inhalator,

a perforated membrane having a plurality of preselected perforated portions each holding and dispensing a reproducible unit dose of less than 50 mg of the said inhalable pwder, said membrane being mounted for movement between said conduit and said storage chamber so that one of said preselected portions is positioned across said conduit whereby the active compound held in the perforation thereof can be dispensed into the conduit and another of said preselected portions thereof is disposed within said storage chamber, dose loading means for introducing said inhalable powder in the storage chamber into the perforation of the preselected portion of said membrane disposed within the storage chamber, and

maneuvering means for displacing the perforated membrane through a plurality of positions whereby successive preselected portions of the perforated membrane holding the inhalable powder are positioned across said conduit for dispensing the inhalable powder,

for the administration of an inhalable powdered containing tiotropium, preferably in an amount of 0.001 to 5%, in admixture with a physiologically acceptable excipient with an average particle size of between 10 to 500 μm.

In yet another preferred embodiment the invention relates to an inhalation kit consisting of an inhalable powdered containing tiotropium, preferably in an amount of 0.001 to 5%, in admixture with a physiologically acceptable excipient with an average particle size of between 10 to 500 μm, and the inhaler according to U.S. Pat. No. 4,524,769, comprising

a nozzle, a conduit connected to the nozzle, a storage chamber adjacent said conduit for storing said inhalable powder to be dispensed by said inhalator, a perforated membrane having a plurality of preselected perforated portions each holding and dispensing a reproducible unit dose of less than 50 mg of the said inhalable pwder, said membrane being mounted for movement between said conduit and said storage chamber so that one of said preselected portions is positioned across said conduit whereby the active compound held in the perforation thereof can be dispensed into the conduit and another of said preselected portions thereof is disposed within said storage chamber, dose loading means for introducing said inhalable powder in the storage chamber into the perforation of the preselected portion of said membrane disposed within the storage chamber, and

maneuvering means for displacing the perforated membrane through a plurality of positions whereby successive preselected portions of the perforated membrane holding the inhalable powder are positioned across said conduit for dispensing the inhalable powder.

In another preferred embodiment according to the invention the inhaler according to U.S. Pat. No. 5,590,645 is applied. The disclosure of U.S. Pat. No. 5,590,645 is incorporated herein by reference in its entirety.

Accordingly, in a preferred embodiment the invention relates to a method for the administration of an inhalable powder containing tiotropium, preferably in an amount of 0.001 to 5%, in admixture with a physiologically acceptable excipient with an average particle size of between 10 to 500 μm, by means of the inhaler according to U.S. Pat. No. 5,590,645, comprising

a medicament pack having a plurality of containers for containing medicament in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale medicament in powder form from such an opened container, and

indexing means for indexing in communication with said outlet containers of a medicament pack in use with said inhalation device.

In another embodiment, the invention relates to a method for treatment of airway diseases, particularly COPD (chronic obstructive pulmonary disease) and asthma, charcterized in that an inhalable powder containing tiotropium, preferably in an amount of 0.001 to 5%, in admixture with a physiologically acceptable excipient with an average particle size of between 10 to 500 μm, is administered via inhalation by the inhaler according to U.S. Pat. No. 5,590,645, comprising

a medicament pack having a plurality of containers for containing medicament in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale medicament in powder form from such an opened container, and

indexing means for indexing in communication with said outlet containers of a medicament pack in use with said inhalation device.

In another preferred embodiment the invention relates to the use of the inhaler according to U.S. Pat. No. 5,590,645, comprising

a medicament pack having a plurality of containers for containing medicament in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale medicament in powder form from such an opened container, and

indexing means for indexing in communication with said outlet containers of a medicament pack in use with said inhalation device,

for the administration of an inhalable powdered containing tiotropium, preferably in an amount of 0.001 to 5%, in admixture with a physiologically acceptable excipient with an average particle size of between 10 to 500 μm.

In yet another preferred embodiment the invention relates to an inhalation kit consisting of an inhalable powdered containing tiotropium, preferably in an amount of 0.001 to 5%, in admixture with a physiologically acceptable excipient with an average particle size of between 10 to 500 μm, and the inhaler according to U.S. Pat. No. 5,590,645, comprising

a medicament pack having a plurality of containers for containing medicament in powder form wherein the containers are spaced along the length of and defined between two peelable sheets secured to each other, an opening station for receiving a container of said medicament pack being, means positioned to engage peelable sheets of a container which has been received in said opening station for peeling apart the peelable sheets, to open such a container, an outlet, positioned to be in communication with an opened container, through which a user can inhale medicament in powder form from such an opened container, and

indexing means for indexing in communication with said outlet containers of a medicament pack in use with said inhalation device.

As described in U.S. Pat. No. 5,590,645 the medicament pack may comprise a flexible strip defining a plurality of pockets each of which contains a dose of medicament which can be inhaled, in the form of a powder. The strip may comprise a base sheet in which blisters are formed to define the pockets, and a lid sheet which is hermetically sealed to the base sheet except in the region of the blisters. The sheets are sealed to one another over their whole width and are preferably formed of a plastics/aluminium laminate. The lid and base sheets are each preferably formed of a plastics/aluminium laminate, and the lid and base sheets are preferably adhered to one another by heat sealing. By way of example, the lid material may be a laminate consisting of 50 gsm bleach kraftpaper/12 micron polyester (PETP) film/20 micron soft temper aluminium foil/9 gsm vinylic peelable heat seal lacquer (sealable to PVC), and the base material may be a laminate consisting of 100 micron PVC/45 micron soft temper aluminium foil/25 micron orientated polyamide. The lacquer of the lid material is sealed to the PVC layer of the base material to provide the peelable seal between the lid and base sheets.

In another preferred embodiement the instant invention is directed to a medicament pack comprising a base sheet in which blisters are formed to define the pockets, and a lid sheet which is hermetically sealed to the base sheet except in the region of the blisters, the sheets being sealed to one another over their whole width and being preferably formed of a plastics/aluminium laminate, wherein the dose of the medicament is an inhalable powder containing tiotropium, preferably in an amount of 0.001 to 5%, in admixture with a physiologically acceptable excipient preferably with an average particle size of between 10 to 500 μm.

In another preferred embodiment according to the invention the inhaler according to U.S. Pat. No. 4,627,432 is applied. The disclosure of U.S. Pat. No. 4,627,432 is incorporated herein by reference in its entirety.

Accordingly, in a preferred embodiment the invention relates to a method for the administration of an inhalable powder containing tiotropium, preferably in an amount of 0.001 to 5%, in admixture with a physiologically acceptable excipient with an average particle size of between 10 to 500 μm, by means of the inhaler according to U.S. Pat. No. 4,627,432, being characterised by a housing with a chamber therein, an air inlet into the chamber,

a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the medicament will be released from the container and entrained in the air flow produced by a patient inhaling through the outlet, and means between said disc and said housing for rotatably indexing the disc to register each of the apertures in turn with the housing opening.

In another embodiment, the invention relates to a method for treatment of airway diseases, particularly COPD (chronic obstructive pulmonary disease) and asthma, charcterized in that an inhalable powder containing tiotropium, preferably in an amount of 0.001 to 5%, in admixture with a physiologically acceptable excipient with an average particle size of between 10 to 500 μm, is administered via inhalation by the inhaler according to U.S. Pat. No. 4,627,432, being characterised by a housing with a chamber therein, an air inlet into the chamber,

a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the medicament will be released from the container and entrained in the air flow produced by a patient inhaling through the outlet, and means between said disc and said housing for rotatably indexing the disc to register each of the apertures in turn with the housing opening.

In another preferred embodiment the invention relates to the use of the inhaler according to U.S. Pat. No. 4,627,432 being characterised by a housing with a chamber therein, an air inlet into the chamber,

a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the medicament will be released from the container and entrained in the air flow produced by a patient inhaling through the outlet, and means between said disc and said housing for rotatably indexing the disc to register each of the apertures in turn with the housing opening,

for the administration of an inhalable powdered containing tiotropium, preferably in an amount of 0.001 to 5%, in admixture with a physiologically acceptable excipient with an average particle size of between 10 to 500 μm.

In yet another preferred embodiment the invention relates to an inhalation kit consisting of an inhalable powdered containing tiotropium, preferably in an amount of 0.001 to 5%, in admixture with a physiologically acceptable excipient with an average particle size of between 10 to 500 μm, and the inhaler according to U.S. Pat. No. 4,627,432, being characterised by a housing with a chamber therein, an air inlet into the chamber,

a circular disc having an axis substantially coaxial to the chamber axis and rotatable inside the chamber and provided with a plurality of apertures therethrough arranged in a circle, said apertures being sized and positioned so that each aperture is adapted to be aligned with a different container, the said disc being arranged so that the carrier can be placed in contact with one face of the disc with one of the containers located in each one of the apertures, an outlet through which a patient may inhale leading out of the chamber, an opening in said housing alignable with respective ones of the apertures in the disc as the disc is rotated, a plunger operatively connected to said housing and having a penetrating member, said penetrating member being displaceable to pass through said opening and the corresponding aperture in the disc registered with it thereby to penetrate and open a container located in the aperture so that the medicament will be released from the container and entrained in the air flow produced by a patient inhaling through the outlet, and means between said disc and said housing for rotatably indexing the disc to register each of the apertures in turn with the housing opening.

The following Examples serve to illustrate the present invention further without restricting its scope to the embodiments provided hereinafter by way of example.

### EXAMPLES

Starting Materials

As a starting material for the synthesis of crystalline tiotropiumbromide monohydrate tiotropiumbromide obtained according to the disclosure of European patent application EP 418 716 A1 is be used.

Preparation of Crystalline Tiotropium Bromide Monohydrate:

15.0 kg of tiotropium bromide as obtained according to the methods disclosed in EP 418 716 A1 are added to 25.7 kg of water in a suitable reaction vessel. The mixture is heated to 80-90° C. and stirred at constant temperature until a clear solution is formed. Activated charcoal (0.8 kg), moistened with water, is suspended in 4.4 kg of water, this mixture is added to the solution containing the tiotropium bromide and rinsed with 4.3 kg of water. The mixture thus obtained is stirred for at least 15 min at 80-90° C. and then filtered through a heated filter into an apparatus which has been preheated to an outer temperature of 70° C. The filter is rinsed with 8.6 kg of water. The contents of the apparatus are cooled at 3-5° C. every 20 minutes to a temperature of 20-25° C. The apparatus is further cooled to 10-15° C. using cold water and crystallisation is completed by stirring for at least one hour. The crystals are isolated using a suction drier, the crystal slurry isolated is washed with 9 litres of cold water (10-15° C.) and cold acetone (10-15° C.). The crystals obtained are dried in a nitrogen current at 25° C. over 2 hours.

Yield: 13.4 kg of tiotropium bromide monohydrate (86% of theory)

The crystalline tiotropium bromide monohydrate thus obtained is micronised by known methods, to bring the active substance into the average particle size which meets the specifications according to the invention.

The DSC diagram of crystalline tiotropium bromide monohydrate shows two characteristic signals. The first, relatively broad, endothermic signal between 50-120° C. can be attributed to the dehydration of the tiotropium bromide monohydrate to produce the anhydrous form. The second, relatively sharp endothermic peak at 230±5° C. can be put down to the melting of the substance. These data were obtained using a Mettler DSC 821 and evaluated with the Mettler STAR software package. These data, like the other values given in the above Table, were obtained at a heating rate of 10 K/min.

The crystalline tiotropium bromide monohydrate thus obtained was characterised by IR spectroscopy. The data was obtained using a Nicolet FTIR spectrometer and evaluated with the Nicolet OMNIC software package, version 3.1. The measurement was carried out with 2.5 μmol of tiotropium bromide monohydrate in 300 mg of KBr. Table 1 shows some of the essential bands of the IR spectrum.

TABLE 1
Attribution of specific bands

| Wave number (cm⁻¹) | Attribution | Type of oscillation |
|---|---|---|
| 3570, 410 | O—H | elongated oscillation |
| 3105 | Aryl C—H | elongated oscillation |
| 1730 | C═O | elongated oscillation |
| 1260 | Epoxide C—O | elongated oscillation |
| 1035 | Ester C—OC | elongated oscillation |
| 720 | Thiophene | cyclic oscillation |

The crystalline tiotropium bromide monohydrate was characterised by X-ray structural analysis. The measurements of X-ray diffraction intensity were carried out on an AFC7R-4-circuit diffractometer (Rigaku) using monochromatic copper K_{α} radiation. The structural solution and refinement of the crystal structure were obtained by direct methods (SHELXS86 Program) and FMLQ-refinement (TeXsan Program). The X-ray structural analysis carried out showed that crystalline tiotropium bromide hydrate has a simple monoclinic cell with the following dimensions: a=18.077 Å, b=11.9711 Å, c=9.9321 Å, β=102.691°, V=2096.96 Å³.

Apparatus

The following machines and equipment, for example, may be used to prepare the inhalable powders according to the invention:

Mixing container or powder mixer: Gyrowheel mixer 200 L; type: DFW80N-4; made by: Messrs Engelsmann, D-67059 Ludwigshafen.

Granulating sieve: Quadro Comil; type: 197-S; made by: Messrs Joisten & Kettenbaum, D-51429 Bergisch-Gladbach.

The following examples provide for inhalable powder mixtures applicable according to the invention.

### Example 1

5.2 kg of glucose monohydrate for inhalation (average particle size 25 μm) are used as the excipient. 22.5 g crystalline tiotropiumbromide monohydrate (micronised; average particle size 1-3.5 μm) are used as the active ingredient.

The aforementioned components are sieved in in alternate layers of lactose monohydrate in batches of about 200 g and crystalline tiotropiumbromide monohydrate in batches of about 1 g. The ingredients sieved in are then mixed together (mixing at 900 rpm).

According to the invention preferably 5.2225 mg of the aforementioned powder are delivered per dose.

### Example 2

5.4775 kg of lactose monohydrate for inhalation (average particle size 25 μm) are used as the excipient. 22.5 g crystalline tiotropiumbromide monohydrate (micronised; average particle size 1-3.5 μm) are used as the active ingredient.

The aforementioned components are sieved in in alternate layers of lactose monohydrate in batches of about 200 g and crystalline tiotropiumbromide monohydrate in batches of about 1 g. The ingredients sieved in are then mixed together (mixing at 900 rpm).

According to the invention preferably 5.5 mg of the aforementioned powder are delivered per dose.

### Example 3

1.1: Excipient Mixture:

5.203 kg of lactose monohydrate for inhalation (average particle size 25 μm) are used as the coarser excipient component. 0.27 kg of lactose monohydrate (5 μm) are used as the finer excipient component. In the resulting 5,473 kg of excipient mixture the proportion of the finer excipient component is 5%.

The aforementioned components are sieved in in alternate layers of lactose monohydrate (25 μm) in batches of about 200 g and lactose monohydrate (5 μm) in batches of about 10 g. The ingredients sieved in are then mixed together (mixing at 900 rpm).

1.2: Final Mixture

To prepare the final mixture, 5,473 kg of the excipient mixture (1.1) and 22.5 g crystalline tiotropiumbromide monohydrate (micronised; average particle size 1-3.5 μm) are used. The content of active substance in the resulting powder is 0.4%.

The aforementioned components are sieved in in alternate layers of excipient mixture (1.1) in batches of about 200 g and crystalline tiotropiumbromide monohydrate in batches of about 1 g. The ingredients sieved in are then mixed together (mixing at 900 rpm).

According to the invention preferably about 5.5 mg of the aforementioned powder are delivered per dose.

The aforementioned tiotropium containing powder mixture is to be stored under conditions that prevent moisture uptake. Preferably, the tiotropium powder blend is packed into receptacles or containers in the form of foil bags which are sealed after filling with tiotropium powder. The foil bag is preferably made of a foil of metal or metal alloy, preferably aluminum, gold or copper, or plastic material, preferably a thermoplastic material. In the alternative, the foil bag is made of a composite foil of plastic material and metal. The composite foil preferably comprises two or three foils which are joined together. The foil bag may further comprise a plastic foil to which a layer of metal, preferably aluminium, glass or ceramic is applied, for example by vapor deposition. The foils of plastic material or metal are several micrometers thick. The thickness of the vapor-deposition layers of metal, glass or ceramic may be in the sub-micrometer range.

Another embodiment of the invention is directed to tiotropium containing receptacles or containers comprising at least one layer of aluminium foil. Most preferred are tiotropium powder containing foil bags which possess at least one layer of aluminium foil. Tiotropium powder stored in such containers proved to be sufficiently protected against moisture uptake.

For the filling of tiotropium powder into the inhalers and medicament packs specified hereinbefore it is recommended to handle the tiotropium powder at the following conditions: 19-25° C. and 15-25% r.h. (relative humidity).

We claim:

1. A receptacle comprising tiotropium or a physiologically acceptable salt thereof and a physiologically acceptable excipient, wherein
the receptacle is able to store the tiotropium under conditions that prevent moisture uptake.

2. The receptacle according to claim 1, wherein the receptacle is a foil bag.

3. The receptacle according to claim 2, wherein the foil bag is made of metal, metal alloy, or plastic material.

4. The receptacle according to claim 3, wherein the metal or metal alloy comprises aluminum, gold or copper.

5. The receptacle according to claim 3, wherein the plastic material is a thermoplastic material.

6. The receptacle according to claim 2, wherein the foil bag is a composite of plastic material and metal.

7. The receptacle according to claim 6, wherein the metal or metal alloy comprises aluminum, gold or copper.

8. The receptacle according to claim 6, wherein the plastic material is a thermoplastic material.
